(19) United States
(12) Patent Application Publication
(10) Pub. No.: US 2018/0142294 A1
(43) Pub. Date: May 24, 2018

(54) METHOD FOR AUTHENTICATING ACTIVE PHARMACEUTICAL INGREDIENTS

(71) Applicant: APDN (B.V.I.) INC., Tortola (VG)

(72) Inventors: James A. Hayward, Stony Brook, NY (US); Michael E. Hogan, Stony Brook, NY (US); MingHwa Benjamin Liang, East Setauket, NY (US); Lawrence Jung, Dix Hills, NY (US)

(21) Appl. No.: 15/800,768

(22) Filed: Nov. 1, 2017

Related U.S. Application Data
(63) Continuation-in-part of application No. 15/553,246, filed on Aug. 24, 2017.
(60) Provisional application No. 62/134,437, filed on Mar. 17, 2015.
(60) Provisional application No. 62/524,186, filed on Jun. 23, 2017.

(51) Int. Cl. C12Q 1/6876; G01N 33/15; G09F 3/02

(52) U.S. Cl. CPC C12Q 1/6876; G09F 2003/0277; G09F 3/02; G01N 33/15

(57) ABSTRACT

Provided is a method of authenticating an active pharmaceutical ingredient (API). The method includes providing an API or an API component and adding a nucleic acid marker having a nucleic acid marker sequence to produce a marked API or a marked API component. The presence of the nucleic acid marker is detected in the sample and the authenticity of the API is thereby determined according to whether the pharmaceutical product includes marked API or the marked API component.

### CROSS-REFERENCE TO RELATED APPLICATIONS

The present application is a continuation of U.S. patent application Ser. No. 15/553,246, filed on Aug. 24, 2017, which claims the benefit of International Application No. PCT/US16/022532, filed Mar. 16, 2016, which claims priority to U.S. Provisional Patent Application No. 62/134,437, filed Mar. 17, 2015. The present application also claims the benefit of U.S. Provisional Application No. 62/524,186, filed Jun. 23, 2017. The contents of the applications listed above are hereby incorporated by reference in their entireties.

### FIELD OF THE INVENTION

The present invention pertains to a method and system for authenticating active pharmaceutical ingredients. More particularly, the present invention pertains to authenticating active pharmaceutical ingredients which are marked with one or more nucleic acid markers.

### BACKGROUND OF THE INVENTION

Authentic pharmaceutical products and medications include one or more active pharmaceutical ingredients (APIs). An API is a composition (or ingredient) in a pharmaceutical product that is biologically active. APIs may be combined with one or more excipients to form a pharmaceutical product. Excipients are substances which are generally inert and are combined with APIs to form pharmaceutical products. Excipients are often referred to as “bulking agents,” “fillers” or “diluents.” In some embodiments, excipients may confer one or more therapeutic benefits on APIs in a pharmaceutical product. For example, excipients may facilitate the absorption or solubility characteristics of a drug, which might not be achieved by the API alone in the pharmaceutical product. Excipients may also be useful in manufacturing of a pharmaceutical product that includes one or more APIs, for instance, by rendering an API soluble, or reducing a change in resistance to flow of the API.

Counterfeit pharmaceutical products and medications represent a worldwide problem. As much as 10% of prescription drugs may be counterfeit according to the World Health Organization (WHO). It has been reported that counterfeit drugs are a $200-billion-a-year industry. Counterfeit or adulterated versions of pharmaceutical products and medications are often substituted for authentic pharmaceutical products or medications, which include one or more intended active pharmaceutical ingredients (APIs). For example, the World Trade Organization (WTO) has indicated that as many as 100,000 people die in Africa each year as a result of consuming counterfeit or adulterated anti-malaria drugs. It has been estimated that Western Europeans spend as much as $14.3 billion annually on illicitly sourced medications, which might not include any of the intended API or may include a lower concentration of the intended API. Thus, a need exists to be able to reliably authenticate an API included in medications and pharmaceutical products.

One method of authenticating APIs is to use a physical or chemical drug formulation identifier (PCID). PCIDs are one or more substances possessing unique physical or chemical properties. PCIDs may be used to identify and authenticate a pharmaceutical product. For example, PCIDs may include inks, pigments, and flavors. PCIDs can be detected by wholesalers, pharmacists, regulators or law enforcement at any point in the supply chain or at any point in the stream of commerce to determine the authenticity of pharmaceutical products.

There remains a need for new technology to verify the authenticity of pharmaceuticals in a manner that cannot be easily designed around by counterfeiters and can be applied as easily in-field as in the laboratory.

### SUMMARY OF THE INVENTION

Exemplary embodiments of the present invention provide a method for authenticating an active pharmaceutical ingredient (API). The method includes providing an API or an API component and adding a nucleic acid marker having a nucleic acid marker sequence to produce a marked API or a marked API component. At least a portion of the marked API or marked API component is incorporated into a pharmaceutical product. A sample of the pharmaceutical product including the marked API or marked API component is obtained. The sample is subjected to an amplification reaction to produce one or more amplification products that are characteristic of the nucleic acid marker. The presence of the nucleic acid marker is detected in the sample and the authenticity of the API is thereby verified indicating that the pharmaceutical product includes the marked API or the marked API component.

The amplification of the sample may be performed by any suitable reaction method. For example, the sample may be amplified by a polymerase chain reaction (PCR). Alternatively, the amplification of the sample may be performed by an isothermal amplification reaction, a rolling circle reaction, a LAMP reaction or the like.

According to an exemplary embodiment of the present invention, the nucleic acid marker may include DNA. The pharmaceutical product may be a tablet, a gel-tab or a capsule. The pharmaceutical product may include a granule or a powder, and the granule or powder may be mixed with one or more liquids to create a suspension or a solution.

The nucleic acid marker may be included in an ink used for printing on a pharmaceutical product, such as a tablet or a capsule. Alternatively, the nucleic acid marker may be included in a dye, which is used to mark a surface of the pharmaceutical product, or which is used as a colorant for the pharmaceutical product. The nucleic acid marker may be included with (e.g. mixed with) an excipient or a diluent that is combined with one or more APIs to form the pharmaceutical product.

Applicants have discovered a new means of using nucleic acids as a taggant in pharmaceutical products to provide rapid information regarding the authenticity and origin of the pharmaceutical products. The invention relates to a method of authenticating a pharmaceutical product including the steps of: adding a detectable nucleic acid marker to a pharmaceutical grade ink to form a tagged ink; marking a pharmaceutical product with the tagged ink; obtaining a sample of the tagged ink on the pharmaceutical product; and detecting the presence of the detectable nucleic acid marker in the ink on the pharmaceutical product, without extraction or purification of the sample, to authenticate the pharmaceutical product.

Preferably, detection is conducted with an in-field nucleic acid detection device.

An emulsifier may optionally be added to the pharmaceutical grade ink with the detectable nucleic acid marker to from the tagged ink. The detectable nucleic acid marker is preferably a detectable DNA marker. Preferably, the DNA marker is added to the ink in an amount ranging from about 10 μg/L to about 10 mg/L or an amount ranging from about 10 fg/L to about 1 μg/L.

The unique DNA sequence of the detectable DNA marker may encode information related to the composition, origin, and/or expiration of the pharmaceutical product. Additionally, the information may include one or more of a production lot number, a date, a time, and a manufacturer.

In one aspect of the invention, the tagged ink consists essentially of the pharmaceutical grade ink and a detectable nucleic acid marker. In another aspect of the invention, the tagged ink consists essentially of the pharmaceutical grade ink, an emulsifier, and a detectable nucleic acid marker.

Preferably, the detectable nucleic acid marker has not been alkaline activated or added to a physical carrier prior to being added to the pharmaceutical grade ink.

The preferred pharmaceutical products are tablets or capsules. Preferably, the tagged ink is present in less than 1×10⁻¹² g per individual tablet or capsule and more than 1×10⁻¹⁸ g per individual tablet or capsule.

Detecting the presence of a nucleic acid marker in the ink on the pharmaceutical product is preferably done using isothermal amplification and a sequence specific detection technique; RPA and an intercalating dye; or PCR-based techniques such as qPCR or qPCR and an intercalating dye. The in-field nucleic acid detection device is preferably an integrated system, a microarray, or a next-generation DNA sequencer.

Another means of detecting the presence of a nucleic acid marker in the ink on the pharmaceutical product is PCR-CE.

### DESCRIPTION OF THE FIGURES

FIG. 1. DNA Tagging and recovery of the Food Grade Ink Applied to a Capsule Methods for marking ink (OPACODE S-1-17823 black) with DNA then applying to acetaminophen capsules

In the top left panel, two ink labeled acetaminophen capsules are displayed. The capsule on the left was DNA marked (with both an “L” and a “5”) by including the DNA into a pharmaceutical ink, then applying the ink via ordinary high speed pharmaceutical capsule pad printing. The matched capsule on the right was marked with the same “L” and “5” but without DNA in the black ink. Both the +DNA and −DNA capsules appear identical to the eye. In the top right panel, it can be seen that the DNA+ink can be swabbed from the surface using ethanol as a wetting solvent. There is no marring of the surface of the capsule in the process. In the lower left panel is an image of the swab after swabbing of the capsule, with the [DNA+ink] complex positioned at the tip. In the lower right panel, the tip of the cotton swab (with DNA containing ink on it) has been cut off and placed into a 0.2 mL tube for DNA amplification via PCR/CE analysis (FIG. 2A), or Isothermal amplification and detection (FIG. 2B), or (in a 0.1 mL tube) for qPCR amplification and detection (FIG. 2C)

FIG. 2. FIGS. 2A-2C Multiple Methods of DNA Amplification and Detection after Capsule Swabbing

FIG. 2A: PCR and CE analysis. The top panel shows, to scale, the equipment required to run the assay: a thermal cycler for amplification (top right) capillary electrophoresis (CE) for size separation and detection (top middle), and a PC for data analysis (top left). The bottom panel displays final output of the analysis by PCR/CE, with clear differentiation between DNA tagged (+DNA) and untagged capsules (−DNA). The DNA of interest is of a known length and sequence and if present in a sample, appears as a single discrete peak of the correct predicted size on such a CE trace. In the (−DNA) capsule, where the “L” and “5” have both been swabbed off for analysis denoted as “−DNA (L+5)”, there is no DNA peak observed at the appropriate area of the CE trace. On the other hand for the DNA labeled capsules (+DNA) where the “L,” “5” or both “L+5” have been sampled, DNA is detected in the expected region of the CE trace (blue peaks) associated with the known DNA length. Long term stability of the DNA ink was also verified as the subject samples were marked with DNA ink more than 2 years prior to the program of swabbing and analysis described in FIG. 2A. The detection of DNA in the DNA+capsule as shown in FIG. 2A confirms long term stability (i.e., greater than 2 years at ordinary air conditioned ambient temperature) of the DNA ink/capsule complex.

FIG. 2B: Isothermal DNA amplification and detection with the Axxin T8-ISO amplification block and detector using TwistDx's RPA-TwistAmp exo real time quantitative isothermal amplification detection chemistry. The top panel depicts an image of the Axxin T9-Iso device along with its dimensions. It is smaller than a typical PCR machine (FIG. 2A). The middle panel depicts representative real-time amplification data derived from the assay as deployed on the intact swab-DNA complex. This data can be accessed onboard the device or exported and viewed on a laptop or PC. As shown, there is a clear differentiation in the amplification curves of the DNA tagged (+DNA) capsules (“L+5” sampling) versus the unmarked (−DNA) capsules (“L+5” sampling), with only minor sample to sample variation. As seen in FIG. 2B, the observable differentiation between tagged and untagged capsules can be observed in as little 10 minutes, but for this assay, since we wanted to show a plateau, it has taken 15 minutes for completion. The bottom panel shows a positive and negative result as a Positive (+) or Negative (−) on the machine display and PC summary, ranked according to the order the sample is loaded onto the machine. This allows the removal of the user interpretation, and allows the machine to have learned a pattern and call a result as-is. As in FIG. 2A, the subject samples in FIG. 2B were marked with DNA ink more than 2 years prior to swabbing and analysis, thus confirming long term stability (i.e., greater than 2 years at ordinary air conditioned ambient temperature) of the DNA ink/capsule complex.

FIG. 2C: Real time, TaqMan qPCR using a MyGo mini device and a Microsoft Surface computer. The top panel depicts an image (to scale) of the MyGo Mini qPCR device next to a Microsoft Surface laptop. The bottom panel depicts a graphical representation of the TaqMan qPCR assay. This can be accessed onboard the device or exported and viewed on a laptop or PC. As depicted here, there is a clear differentiation in the amplification curves between the DNA tagged (+DNA) capsules versus the non-DNA tagged capsules (−DNA) with minor sample to sample variation. In all cases both the “L” and “5” symbols have been swabbed. As seen here, the differentiation between tagged and untagged is clearly observable by 30 cycles (40 minutes) but since the inventors wanted to show a plateau, it has been taken 45 cycles completion. The software generates a table from such data where a human interpreter is required to interpret the sample as containing DNA. An algorithm may be used to remove the need for human interpretation. As in FIGS. 2A & 2B, the subject samples in FIG. 2C were marked with DNA ink more than 2 years prior to the swabbing and analysis, thus confirming long term stability (i.e., greater than 2 years at ordinary air conditioned ambient temperature) of the DNA ink/capsule complex.

### DETAILED DESCRIPTION

Exemplary embodiments of the present invention provide a method for authenticating an active pharmaceutical ingredient (API). A system and method for authenticating tablets is described in U.S. Pat. No. 8,420,400 to Hayward et al., the disclosure of which is hereby incorporated by reference in its entirety.

Exemplary embodiments of the present invention provide a method for authenticating an active pharmaceutical ingredient. The method includes providing an API or an API component and adding a nucleic acid marker having a nucleic acid marker sequence to produce a marked API or a marked API component. At least a portion of the marked API or marked API component is incorporated into a pharmaceutical product. A sample of the pharmaceutical product including the marked API or marked API component is obtained. The sample is subjected to an amplification reaction to produce one or more amplification products that are characteristic of the nucleic acid marker. The presence of the nucleic acid marker is detected in the sample and the authenticity of the API is thereby determined, indicating to whether the pharmaceutical product includes marked API or the marked API component.

The amplification of the sample may be performed by any suitable reaction method. For example, the sample may be amplified by a polymerase chain reaction (PCR). Alternatively, the amplification of the sample may be performed by an isothermal amplification reaction, a rolling circle reaction, a LAMP reaction, Multiple Annealing and Loop based amplification (MALBAC), Strand Displacement amplification (SDA), Nicking Enzyme amplification reaction (NEAR), Recombinase Polymerase amplification (RPA), Helicase dependent amplification (HDA), Thermal Helicase dependent amplification (tHDA), Loop Mediated isothermal amplification (LAMP), or the like.

According to an exemplary embodiment of the present invention, the nucleic acid marker may include DNA. The pharmaceutical product may be a tablet, a gel-tab or a capsule. The pharmaceutical product may include a granule or a powder, and the granule or powder may be mixed with one or more liquids to create a suspension or a solution.

The nucleic acid marker may be included in an ink used for printing on a pharmaceutical product, such as a tablet or a capsule. Alternatively, the nucleic acid marker may be included in a dye, which is used to mark a surface of the pharmaceutical product, or which is used as a colorant for the pharmaceutical product. The colorant may be used to form a colored coating for the pharmaceutical product or may be used to color the entire pharmaceutical product. The colored coating may be used to visually identify the pharmaceutical product. The nucleic acid marker may be included with (e.g. mixed into) an excipient or a diluent that is combined with one or more APIs to form the pharmaceutical product.

In one embodiment, the invention relates to a method of marking a pharmaceutical product and then authenticating the pharmaceutical product. The pharmaceutical product may be any pharmaceutical (drug/dosage) that can be marked with ink. For example, the pharmaceutical product may be a tablet or a capsule.

The steps of marking the pharmaceutical product for authentication include adding a detectable nucleic acid marker to a pharmaceutical grade ink to form a tagged ink and marking a pharmaceutical product with the tagged ink. Depending upon the solvent composition of the ink, the nucleic acid marker may be added directly to the ink to form a solution. For example in the case of primarily water based inks, the nucleic acid marker may be added directly to the ink. For a primarily non-aqueous ink (as in the case of FIGS. 2A-2C), the nucleic acid is added to the ink in the presence of an emulsifier to form a stable emulsion. Preferred emulsifiers are polyethylene glycol (PEG) or an equivalent.

### Active Pharmaceutical Ingredients (APIs)

API's may be included in pharmaceutical products, which may be in the form of tablets, powders, suspensions, liquids (e.g., injectables) and inhalants. For example, pharmaceutical products include injectable, topical, or pulmonary (e.g. an inhaled vapor or an inhaled powder) products. APIs may be included in pharmaceutical products, such as medicines, which appear in many forms such as tablets, capsules, gel-tabs, oral liquids, topical creams and gels, transdermal patches, injectables, implants, eye products, nasal products, inhalers and suppositories.

The nucleic acid marker, described in more detail below, may be used to mark bulk APIs. For example, the nucleic acid marker may be used to mark the bulk API before the bulk API is combined with any excipients. The ability to mark bulk APIs before being combined with excipients allows APIs to be identified at any point in the supply chain, because bulk APIs may be produced off-shore and later imported to the United States or elsewhere for further processing and manufacturing of pharmaceutical products. Further, marked APIs may be detectable by law enforcement agencies to track the movement of counterfeit or adulterated drugs at any point in the stream of commerce.

### Physical or Chemical Drug Formulation Identifier (PCID)

One example of a PCID is a pharmaceutical grade ink or dye which may be printed onto a pharmaceutical product to produce a marked pharmaceutical product. For example, the ink may be printed onto the surface of a tablet, a gel-tab or a capsule. The ink may include one or more identifiers which may be detected to determine the authenticity or counterfeit nature of the marked pharmaceutical product.

According to exemplary embodiments of the present invention, the pharmaceutical grade ink may include one or more nucleic acid markers which are described in more detail below. The pharmaceutical grade ink may include an optional additional PCID such as a marker dye.

The PCID may include inks, pigments, flavors or any other suitable identifier which is added to the pharmaceutical product to identify the authenticity of the pharmaceutical product. For example, inks, pigments and flavors may be combined with the nucleic acid markers described below in more detail and added to pharmaceutical tablets or capsules to identify authentic tablets or capsules. The PCID may serve as a marker for the location of the nucleic acid marker on the pharmaceutical product. For example, the location of ink, which includes the nucleic acid marker and is printed on the pharmaceutical product may indicate the location of the nucleic acid marker on the pharmaceutical product

### In-Field-Authentication of Marked Pharmaceutical Products

According to an exemplary embodiment, the method includes providing a sample from the pharmaceutical product and analyzing the sample to detect the presence of the nucleic acid marker. The analysis is performed using an in-field detection instrument. The in-field detection instrument includes a microsystem configured to perform sample in-answer out analysis. The presence of the nucleic acid marker is detected in the sample and the authenticity of the API is thereby determined according to whether the pharmaceutical product includes marked API or the marked API component.

According to an exemplary embodiment, a kit for collecting the sample from the pharmaceutical product includes a sample collection unit configured to collect a sample including the nucleic acid marker suitable for analysis in an in-field detection instrument. The kit may include a buffer or a solvent suitable for extracting the nucleic acid marker from the pharmaceutical product.

In-field detection of nucleic acid markers is described in more detail in U.S. patent application Ser. No. 14/471,722 filed on Aug. 28, 2014, the disclosure of which is hereby incorporated by reference in its entirety.

In-field detection instruments useful in the invention may include an integrated system with sample in-answer out analysis capability. The integrated system may be a self-contained unit that performs all necessary analysis processes without the need for additional lab equipment. The integrated system may be automated, and may only require the addition of the sample to the integrated system and activation of the integrated system to perform the analysis.

The in-field detection instrument may be portable or fixed in a single location. Sample in-answer out analysis refers to the ability of the integrated system to perform all analysis steps after transferring the sample to the integrated system and automatically providing a result, thus limiting human error from the data interpretation. The integrated system may be configured to provide detection with a minimum level of monitoring or adjustment by the operator. In an exemplary embodiment, the sample is loaded directly or from a sample collection device (such as a swab) configured to mate with a sample port of the integrated system. The integrated system is then activated and the instrument provides detection/authentication data without further operator interaction.

Detection/authentication data may be stored and/or exported. In the case of detecting the presence of a distinctive marker, a sample suspected of including the distinctive marker may be provided and transferred to an in-field detection instrument, and the instrument may automatically determine whether or not the distinctive marker is present in the sample. Exemplary integrated systems for in-field DNA authentication of pharmaceutical products are described in more detail below and in the Description of the Figures.

The in-field detection instrument may communicate with a server comprising authenticity data for the article of interest. The server may comprise an authenticity database storing profile information for a number of distinctive markers associated with a number of articles of interest. Authenticity data may be a unique profile corresponding to the distinctive marker. For example, the distinctive marker may include one or more unique nucleic acid sequences, and the authenticity data may be a digital copy of the unique nucleic acid sequences. The data from the in-field detection instrument may be compared by a remote server to known authenticity data to ascertain the authenticity of the sample being tested. This comparison data may be communicated to the in-field detection instrument and conveyed to the user as “Pass” “No Pass” information displayed on the in-field detection instrument.

### Nucleic Acid Markers

Nucleic acids are particularly well-suited for pharmaceutical products due to their enormous coding capacity. Useful information that can be readily encoded in nucleic acid detectable markers include for example and without limitation: the product production lot number, the date of manufacture or processing, the time of manufacture, the identity of the manufacturer, intended geography of sale, the expiration of the product, and the composition of the product.

Nucleic acids are also ideal as detectable markers for pharmaceutical products because of the fact they can be used in such minute quantities that their sequences are impossible to duplicate without knowledge of their nucleotide sequences or access to a complementary probe or specific primer sequences necessary for their amplification and hence their detection.

The nucleic acid can include RNA, DNA, an RNA-DNA molecule or complex, single stranded DNA or double stranded DNA. The nucleic acid can be any suitable size, for example, the nucleic acid can be in a size range of about 10 base pairs to about 1000 base pairs. The nucleic acid can include any suitable natural or non-natural DNA sequence, such as, for example, a synthetic DNA sequence that is a non-natural DNA sequence. The non-natural DNA sequence can be formed by digesting and re-ligating naturally or non-naturally occurring DNA. The DNA can be from any source, such as, for instance, animal or plant DNA. The nucleic acid can include a non-naturally occurring DNA sequence formed by digesting and re-ligating DNA. The detectable marker can include one or more non-natural nucleic acid sequences derived from any genomic DNA, such as nuclear DNA, mitochondrial DNA or chloroplast DNA. Non-natural DNA can be produced by any method that rearranges the nucleotide sequence, such as the following method. Natural DNA is digested by a restriction enzyme binding to a double stranded DNA molecule and cleaving the double stranded DNA molecule. One or more restriction enzymes are selected that bind a recognition sequence and cleave DNA at the recognition sequence. Suitable recognition sequences include four or six base pairs. Restriction enzymes are selected that bind and cleave DNA to form DNA fragments with “sticky ends.” A sticky end is a stretch of unpaired nucleotides at a terminal end of a DNA fragment. The unpaired nucleotide sequence sticky end of a first DNA fragment binds with a complementary unpaired nucleotide sequence sticky end of a second DNA fragment. Cleaved DNA fragments with sticky ends are ligated (using a DNA ligase) with other cleaved DNA fragments with the same sticky ends (i.e., produced by the same restriction enzyme) to form non-natural DNA with a non-naturally occurring nucleic acid sequences. Many cleaved DNA fragments with sticky ends may be randomly re-ligated to form a new “random” nucleic acid sequence.

In exemplary embodiments, when the nucleic acid marker includes DNA, the DNA may be added to the liquid, tablet or capsule pharmaceutical product in a concentration range of from about 1 ng/L to about 1 μg/mL of DNA in a pharmaceutical product.

The preferred detectable nucleic acid marker is DNA. Any suitable DNA marker may be used in the methods of the present invention. The DNA may be single or double stranded DNA. In one embodiment, the detectable marker DNA may be from about 20 bases to about 700 kilobases in single strand length, or about 20 base pairs to about 700 base pairs in double strand length. The FDA and WHO have both provided guidance that DNA in a certain restricted size range are too short to pose any risk of delivering unexpected biological activity, i.e., they are too short to be a gene. See the Examples for further discussion of FDA and WHO guidelines.

The detectable marker DNA having a unique nucleotide sequence may be included with an excess of a carrier nucleic acid of a natural genomic sequence or a mixture of random synthetic or natural nucleic acid sequences. In preferred embodiments, the carrier DNA is of similar length to the detectable marker DNA having a unique nucleotide sequence. In this way, extraction of total nucleic acid will not reveal the detectable marker DNA sequence without access to the cognate PCR primer pair or pairs for PCR, or the complementary nucleotide hybridization probe depending on the detection method used.

Suitable amounts of detectable marker DNA for incorporation into the pharmaceutical grade ink according to the present invention can range from about 10 fm/L to about 10 mg/L added per liter of ink, with preferred ranges of about 10 μg/L to about 1 mg/L of detectable marker DNA added per liter ink; about 10 μg/L to about 10 mg/L of detectable marker DNA added per liter ink; and about 10 fg/L to about 1 μg/L of detectable marker DNA added per liter ink.

### Fluorescent Markers

The nucleic acid marker may be combined with one or more fluorescent markers to visually detect the presence or location of a nucleic acid marker on a marked pharmaceutical product. Fluorescent markers are described in more detail in U.S. patent application Ser. No. 14/471,722 filed on Aug. 28, 2014, the disclosure of which is hereby incorporated by reference in its entirety.

In some exemplary embodiments of the present invention, marking the pharmaceutical product includes marking the pharmaceutical product or primary and/or secondary packaging of the pharmaceutical product with visual or machine-detectable reporters. The methods of authentication comprise placing, associating, or integrating an optical reporter taggant with the pharmaceutical product or the packaging. The optical reporters can be detected by using a high energy light source for excitation, with the location of nucleic acid marker identified by the presence of an optical reporter. The location and emission wavelength of the optical reporters provides a first level of security or authentication of the labeled pharmaceutical product or packaging. After the location of the optical reporters and associated nucleic acid marker on the pharmaceutical product or packaging has been determined, the nucleic acid marker may be characterized and identified to further increase the level of security and/or authenticity of the pharmaceutical product. When the nucleic acid marker included with the optical reporter is a DNA molecule, PCR or another sequence analysis technique can be utilized to further authenticate the pharmaceutical product.

According to an exemplary embodiment of the present invention, the optical report may include an upconverting phosphor particle (UCP). In some exemplary embodiments, the upconverting phosphor particle UCP is coated with a silylination composition which is configured to covalently link to the nucleic acid marker. UCPs are described in more detail in U.S. Pat. No. 8,420,400 to Hayward et al., the disclosure of which is hereby incorporated by reference in its entirety.

### Excipients

Excipients are substances which are generally inert and are combined with APIs to form pharmaceutical products. Excipients are often referred to as “bulking agents,” “fillers” or “diluents.” Excipients may confer one or more therapeutic benefits on APIs in a pharmaceutical product. For example, excipients may facilitate the absorption or solubility characteristics of a drug, which might not be achieved by the API alone in a pharmaceutical product. Excipients may also be useful in manufacturing of the pharmaceutical product that includes one or more APIs, for instance, by rendering an API soluble, or modifying a resistance to flow of the API. The nucleic acid marker may be combined with excipients included in the pharmaceutical product to authenticate the API included in the pharmaceutical product.

According to an exemplary embodiment of the present invention, the nucleic acid marker may be used to mark a cellulosic excipient, which may be combined with one or more APIs as a bulk filler. Bulk fillers, such as the cellulosic excipient, are commonly used in drug tablets, commercial binders and enteric coatings. Enteric coatings may be used for holding prescription and over-the-counter (OTC) drug tablets together. Excipients may include hydroxypropylcellulose, hydroxyethylcellulose, sodium carboxymethylcellulose, ethylcellulose, microcrystalline cellulose, lactose powder, sucrose powder, and/or cassava flour.

Excipients may be included in pharmaceutical products, such as medicines, which appear in many forms such as tablets, capsules, gel-tabs, oral liquids, topical creams and gels, transdermal patches, injectables, implants, eye products, nasal products, inhalers and suppositories.

Excipients may include diluents, which may be used to provide bulk or to enable dosing of a pharmaceutical product. For example, diluents may include sugar compounds, such as lactose, dextrin, glucose, sucrose or sorbitol.

Excipients may include binders, compression aids, or granulating agents, which may bind tablet ingredients together or provide mechanical strength to the tablet. For example, binders may include natural or synthetic polymers, such as starches, sugars, sugar alcohols and cellulose derivatives.

Excipients may include disintegrants, which may assist in tablet dispersion in the gastrointestinal tract. For example, disintegrants may include starch and cellulose derivatives.

Excipients may include glidants, which may reduce friction in powders and increase adhesion between particles during manufacturing. Glidants may include colloidal anhydrous silicon or silica compounds

Excipients may include lubricants, which may slow disintegration and dissolution of the pharmaceutical product. Lubricants may include stearic acid or stearic acid salts, such as magnesium stearate.

Excipients may include tablet coatings and films, which may protect the tablet from light, air and moisture, which may increase mechanical strength of the tablet and may mask a taste and smell of the tablet. Coatings may also be used to modify an amount of time to release the API from the tablet. Coatings and films may include sugar, or natural or synthetic polymers. For example, cellulose acetate phthalate may be used as an enteric coating to delay release of the API from the tablet.

Excipients may include colorants or coloring agents. Coloring agents may assist in identifying the pharmaceutical product. Colorants and coloring agents may include dyes, such as synthetic dyes, or natural pigments, such as pigments used to color food. According to an exemplary embodiment of the present invention, the nucleic acid marker may be added to a colarant or coloring agent used to confer color on the pharmaceutical product or used to form a color coating on the outside of the pharmaceutical product.

### Pharmaceutical Grade Ink

Any pharmaceutical grade ink may be used. As described above, the nucleic acid marker is added to the ink in such small quantities so that the composition and the stability of the ink is not compromised. The ink and the detectable nucleic acid marker do not need any additional preparation before being mixed together to form the nucleic acid tagged ink. Specifically, the detectable nucleic acid marker does not need to be alkaline activated or chemically modified in any other way. In certain embodiments, an emulsifier may be added to the pharmaceutical grade ink with the detectable nucleic acid marker as part of the formulation process to allow the detectable nucleic acid marker to be delivered as a stable emulsion into a non-aqueous ink. However, generally speaking a perturbant (i.e., a substance that facilitates recovery of the nucleic acid taggant from the ink) does not need to be incorporated into the DNA tagged ink. Experimentation has shown that the pharmaceutical grade inks are generally found to release DNA readily upon swabbing with water, ethanol, or other solvents.

Accordingly, in one embodiment, the tagged ink “consists essentially of” the pharmaceutical grade ink and the detectable nucleic acid marker. In another embodiment, the tagged ink “consists essentially of” pharmaceutical grade ink, the detectable nucleic acid, and an emulsifier. As provided herein, the transitional phrase “consists essentially of” or “consisting essentially of” excludes all items in the ink that materially change the basic and novel characteristics of the tagged ink or its primary components, the pharmaceutical grade ink and the detectable nucleic acid marker. Items that may materially affect the basic and novel characteristics of the tagged ink (and would thereby be excluded by “consisting essentially of” language) include fibers or other physical carriers to which the detectable nucleic acid marker may be attached or associated with; perturbants; and any other type of marker besides the detectable nucleic acid marker. For example, a cyanoacrylate marker would be excluded by the “consisting essentially of” language.

The term “consisting essentially of” would permit the inclusion of compounds that do not materially change the basic and novel characteristics of the tagged ink or its primary components such as dyes, colorants, water, and solvents meant to do no more than alter the viscosity or spreadability of the ink such as food grade/pharmaceutical grade surfactants.

The detectable nucleic acid marker is added to the pharmaceutical grade ink by any method known in the art that will not compromise the stability of the nucleic acid marker or the ink. The preferred method includes mixing at room temperature.

The step of marking the pharmaceutical product with the tagged ink may occur by any method in the art. The ink is usually placed in a printer and the printer will “write” the desired letters or image onto the pharmaceutical product. The ink is then allowed to cure on the pharmaceutical product. Drying times and conditions can be determined by a person having ordinary skill in the art. In one embodiment, the ink is printed via an inkjet/continuous injection printer or a rotogravure printer.

The tagged ink is present in the individual pharmaceutical product (tablet or capsule) at less than 1×10⁻¹² g per tablet/capsule, which is roughly 10⁻⁸ fold less than the level of incidental DNA in a capsule or tablet which the FDA has already determined to be safe.

### Quantitative Detection of Nucleic Acid Markers in APIs

At some time post-curing, the presence of the nucleic acid marker in the tagged ink on the pharmaceutical product may be detected, as set forth herein, to authenticate the pharmaceutical product.

The presence of the nucleic acid may be determined by first obtaining a sample of the tagged ink. For example, a solvent placed on a cotton swab may be used to wipe the ink to obtain the sample. Preferred solvents include water, ethanol, isopropanol, and methyl ethyl ketone. The sample may then be analyzed by any method in the art without the need for DNA isolation, i.e., extraction and purification. Typically before DNA detection techniques can be employed, the DNA in the sample must be isolated and purified to allow for accurate results. Often, the steps of DNA isolation and purification are time consuming and/or costly, and add complexity to the authentication process. The exclusion of these steps greatly simplifies the process of authentication and also reduces the time necessary to authenticate. In the case of PCR, isolation and purification steps are usually required before the DNA can be amplified. The present methods do not require these extra steps of preparing the DNA by isolation, extraction, and/or purification. The new methods allow for results to be obtained quickly and accurately.

Preferred DNA detection analysis methods include PCR-CE (polymerase chain reaction-capillary electrophoresis) or PCR then DNA sequencing or isothermal amplification (such as recombinase polymerase amplification (RPA)) followed by hybridization probe analysis or sequencing. The sample may also be analyzed in the field by PCR-based quantitative methods (such as real time quantitative PCR (qPCR)) or RPA based methods, or similar methods of DNA amplification and detection by thermal cycling or isothermal amplification. Both RPA and qPCR analyses may be performed with an intercalating dye such as SYBR® Green, SYBR® Gold, etc. Next-generation DNA sequencing (high-throughput sequencing) may also be employed. Next-generation DNA sequencing methods allow for quick and inexpensive sequencing. Some examples of next-generation DNA sequencing include, but are not limited to, Illumina (Solexa) sequencing, Roche 453 sequencing, Ion torrent: Proton/PGM sequencing, and SOLiD sequencing. A microarray may also be used as an in-field DNA detection device. In addition, preferred DNA detection analysis methods include any known field-deployable method of DNA detection, whether amplification based, sequence specific based, or both.

According to an exemplary embodiment of the present invention, the amount of nucleic acid marker included in the pharmaceutical product may be quantitatively determined. A predetermined amount of nucleic acid marker may be included in the marked pharmaceutical product. The predetermined amount of nucleic acid marker may be determined with respect to the amount of API and/or the amount of other excipients included in the pharmaceutical product. When the sample is obtained from the pharmaceutical product, an amount of nucleic acid marker included in the sample may be determined and compared with the expected amount of nucleic acid marker based on the initial predetermined amount of nucleic acid marker included in the pharmaceutical product.

In exemplary embodiments, when the nucleic acid marker includes DNA, the DNA may be added to the liquid, tablet or capsule pharmaceutical product in a concentration range of from about 1 ng/L to about 1 μg/mL of DNA in a pharmaceutical product.

According to an exemplary embodiment of the present invention, if the nucleic acid marker is added to the pharmaceutical product in an amount of 100 molecules of nucleic acid marker per dose of the pharmaceutical product, then a sample of the marked pharmaceutical product would be expected to include 100 molecules of nucleic acid marker per dose of the pharmaceutical product. If the amount of nucleic acid marker detected in the pharmaceutical product is less than 100 molecules per dose of the pharmaceutical product, than this may indicate that the pharmaceutical product has been adulterated or tampered with. For example, if the amount of nucleic acid marker detected in the pharmaceutical product is found to be 10 molecules of nucleic acid marker per dose of pharmaceutical product, than this would indicate a 10-fold dilution of the pharmaceutical product.

According to exemplary embodiments of the present invention, as few as 10 molecules of nucleic acid marker per dose of pharmaceutical product may be reliably detected, and may be used to authenticate a pharmaceutical produce that is marked with the nucleic acid marker.

The exemplary embodiments described herein may similarly be applied to food and cosmetic products.

The disclosures of each of the references, patents and published patent applications disclosed herein are each hereby incorporated by reference herein in their entireties.

Having described exemplary embodiments of the present invention, it is further noted that it is readily apparent to those of ordinary skill in the art that various modifications may be made without departing from the spirit and scope of the present invention.

### EXAMPLES

Applicants conducted a study of the DNA tagging of pharmaceutical grade ink with detectable nucleic acid markers. In this study, the detectable nucleic acid marker was DNA. A DNA concentrate was inoculated into one liter of a well-known food grade pharmaceutical ink (OPACODE S-1-17823 black ink) and run through a R. W. Hartnett, Model B-15-8 high speed pad printer for direct printing onto Acetaminophen capsules. Matched samples of un-marked Acetaminophen capsules were purchased from local pharmacies. There was no difference in appearance between the two capsule types (+/−DNA tagging).

Data was obtained two years after completion of the 2014 labeling run, on tablets which had been stored continuously at lab ambient temperature (@25° C.).

In the present pilot study, the DNA tag was introduced into the food grade ink at a mass ratio of 1×10⁻⁶ grams per liter, with PEG (at 5 g/liter) as a DNA emulsifier (See Table 1 below).

TABLE 1
Composition of Standard Pharmaceutical Grade
Opacode S-1-17823 Ink with Added ADNAS DNA
PCID to Label the Exterior of Capsules

| | Bulk link | | Mass applied | |
|---|---|---|---|---|
| Composition of | composition: | | per tablet: | |
| the food grade ink | Mass/liter | | assuming 10⁷ tablets | |
| (Opacode S-1-17823) | of ink stock | | printed per liter | |
| Shellac glaze | 450 | g/liter | 45 × 10⁻⁶ | g |
| Iron oxide (black colorant) | 3 | g/liter | 0.3 × 10⁻⁶ | g |
| Ammonium hydroxide | 208 | g/liter | 28 × 10⁻⁶ | g |
| Propylene Glycol | 250 | g/liter | 25 × 10⁻⁶ | g |
| PEG | 5 | g/liter | 0.5 × 10⁻⁶ | g |
| ADNAS DNA Tag | 10⁻⁶ | g/liter | 1 × 10⁻¹³ | g |

Residual PCR Reactants***

| | | | | |
|---|---|---|---|---|
| Magnesium | 0.14 × 10⁻⁷ | g/liter | <<0.14 × 10⁻¹⁴ | g |
| Tris/HCl | 1.2 × 10⁻⁷ | g/liter | <<1.2 × 10⁻¹⁴ | g |
| Potassium Chloride | 3.75 × 10⁻⁷ | g/liter | <<3.75 × 10⁻¹⁴ | g |
| PCR Primers | 3.75 × 10⁻¹⁰ | g/liter | <<3.75 × 10⁻¹⁷ | g |
| Nucleotide Triphosphates | 0.4 × 10⁻⁷ | g/liter | <<4 × 10⁻¹⁰ | g |
| Taq Polymerase | 3 × 10⁻¹⁰ | g/liter | <<3 × 10⁻¹⁷ | g |
| (recombinant) | | | | |

Notes:
***The amount of residual PCR reactant shown here represents a 100-fold reduction relative to that in the PCR product used for tagging the ink. That reduction is based on the fact that, following the PCR reaction, the DNA had undergone a 2-step column purification, via anion exchange chromatography and then desalting via size exclusion chromatography. That purification procedure is expected to reduce the residual concentration of non-amplicon reactants by at least 100-fold.

SUPPLEMENTARY TABLE 1
The composition of the Master Mix for the
Polymerase Chain Reaction Assay

| TwistAmp exo assay reaction mix component | Per Tube (μL) |
|---|---|
| Extract-N-Amp PCR ReadyMix ™ | 10.0 |
| Extraction Solution | 2.0 |
| Dilution Solution | 2.0 |
| 10 μM Forward Primer | 0.5 |
| 10 μM Reverse Primer | 0.5 |
| PCR PCR Certified Water H₂0 | 3 |
| 25 mM MgCl₂ | 2 |
| Total Volume | 20 |

Given that the DNA ink itself comprises only about 10⁻⁷ of the overall mass of the tablet, the DNA/tablet mass ratio is 10⁻¹³. The FDA and WHO both teach that DNA may be included into an oral dose at up to 100 μg. Thus, the added DNA in the present case is at 10⁻⁹ fold below the body of FDA and WHO guidance, and is specifically below the FDA Guidance provided in, “Industry Incorporation of Physical Chemical Identifiers into Solid Oral Dosage Form Drug Products for Anticounterfeiting,” wherein DNA is recognized as a viable Physical and Chemical Identifier (PCID) for pharmaceutical products. In addition, the DNA used to tag the Acetaminophen capsules in the instant study was of a known length and sequence under 200 bp in length with no chemical modifications, and thus, consistent with present FDA DNA safety guidelines.

The present DNA can be sampled and authenticated via swabbing of the ink with an ethanol-moistened swab, followed by direct analysis of the swab-DNA complex (without DNA isolation or purification of any kind), via laboratory scale PCR-CE (see FIG. 2A). The lower right panel of FIG. 2A demonstrates that the DNA amplicon matching the known length of the DNA tag is readily detectable via PCR-CE in the DNA marked capsules via swabbing the “L” symbol or both the “L” and “5” symbols on the capsule surface. As expected, DNA is not detected in the unmarked tablet control. Importantly, since sampling and analysis was performed after 2 years of tablet storage at 25° C., the data demonstrate that the ambient-temperature shelf life of the DNA tag, as assessed by PCR-CE, is greater than 2 years.

In addition to highly standardized DNA testing such as PCR-CE, in order to monitor a supply chain as complex as that in pharmaceutics, it is necessary to provide methods to sample and rapidly detect the DNA molecular tag in the field. Here, the DNA tag of known length and sequence is detected via two different methods of field-deployable nucleic acid analysis: isothermal amplification with hybridization probe analysis and qPCR with hybridization probe analysis.

Isothermal DNA amplification is now widely deployed as a method of nucleic acid analysis in pathogen testing. FIG. 2B summarizes such field deployable DNA sampling and detection of the known length and sequence DNA tag in the Acetaminophen pilot study: via the sequence specific isothermal DNA amplification chemistry from TwistDx (Alere Corporation). The Twist chemistry was deployed in the context of a portable device (Axxin T8-ISO, Axxin Pty Ltd) which can process up to 8 samples in parallel. The readout from this device can be displayed as a real-time amplification curve or as a simple “plus”/“minus” on the device. Both types of data are exportable to a laptop or PC and then to the internet to support data archiving and additional data analysis. In addition to portability, the instrument also has an open tangential optical path which makes it suitable for direct analysis of swabs and other solid materials.

Using the combination of Twist chemistry and the Axxin device, it is seen (FIG. 2B) that the same process of direct capsule swabbing (without isolation or purification) followed directly by analysis of the swab-DNA complex can cleanly detect the DNA tag in under 20 minutes. During this same time period, no DNA is detected in the unmarked control capsule. Since sampling and analysis for isothermal amplification was also performed after 2 years of tablet storage at 25° C., the data demonstrate that the ambient temperature shelf life of the DNA tag, as assessed by sequence selective Twist isothermal amplification, is also greater than 2 years.

qPCR is also now widely deployed as a method of nucleic acid analysis in pathogen testing. FIG. 2C displays an alternative approach to field-deployed DNA analysis, using instead the industry-standard sequence selective TaqMan qPCR assay (Thermo-Fisher-ABI) as deployed on a small, portable qPCR device, the MyGo Mini (from IT-IS Life Science Ltd) which can process 16 samples in parallel. The readout from the MyGo device is displayed as a real-time amplification curve on a laptop or PC, with a standard analysis time of about 60 min. The qPCR readout from the MyGo device can be displayed as a real-time amplification curve or a table of values on device PC, laptop, or a tablet and are exportable on to the internet, to support data archiving and additional data analysis. In addition to portability, the instrument also has an open tangential optical path which makes it suitable for direct analysis of swabs and other solid materials.

Using the combination of the TaqMan chemistry and the MyGo device, it is seen (FIG. 2C) that the same process of direct swabbing (without isolation or purification) followed directly by the analysis of the unprocessed swab-DNA complex can cleanly detect the known length and sequence DNA tag in the capsules in 60 minutes. During this same time period, no DNA is detected in the unmarked control capsule. Since sampling and analysis for TaqMan qPCR amplification was also performed after 2 years of tablet storage at 25° C., these data demonstrate that the ambient temperature shelf life of the DNA tag, as assessed by the qPCR is also greater than 2 years.

The data presented here show that a PCR generated DNA fragment may be used as a POD (Physical Chemical Identifier) when added to an ordinary pharmaceutical capsule formulation as part of the food grade ink used as part of the capsule coating. The data show that, after 2 years of continuous storage at lab ambient temperature, the DNA tag, although introduced at only 1 ppM into the ink, can be collected by simple surface swabbing, then without subsequent DNA processing, the intact swab-DNA complex can be analyzed by well-known methods of regulated lab based DNA forensics (PCR-CE) and also by the portable methods being developed for food safety, environmental screening and point of care diagnostics (isothermal amplification and qPCR).

The data suggests that DNA tagging can now become a routine component of pharmaceutical supply chain analysis: the goal being to augment better known print-based methods (like serialized bar coding) with the addition of DNA as part of the ink to secure the authenticity of a drug formulation from the manufacturer to the distributor to the pharmacy.

### Methods

### Samples

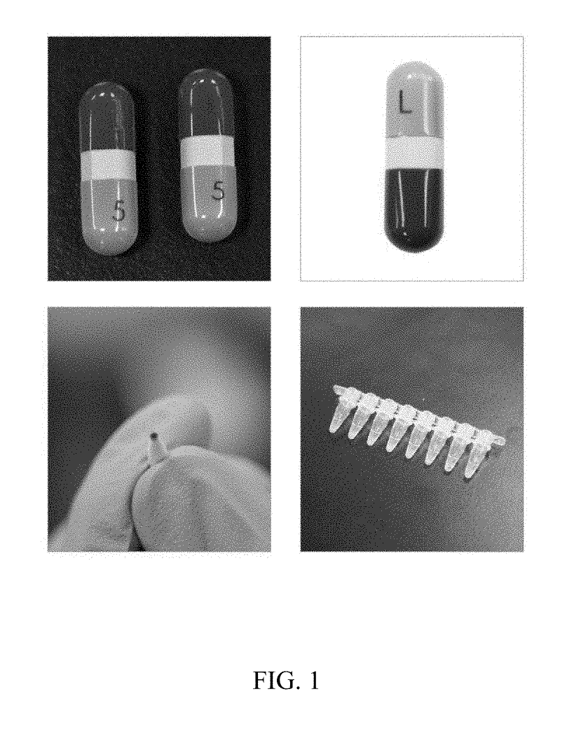

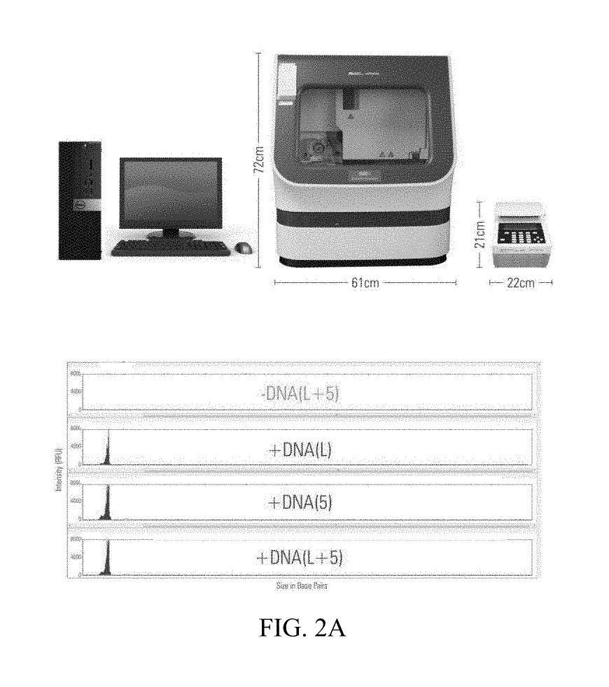

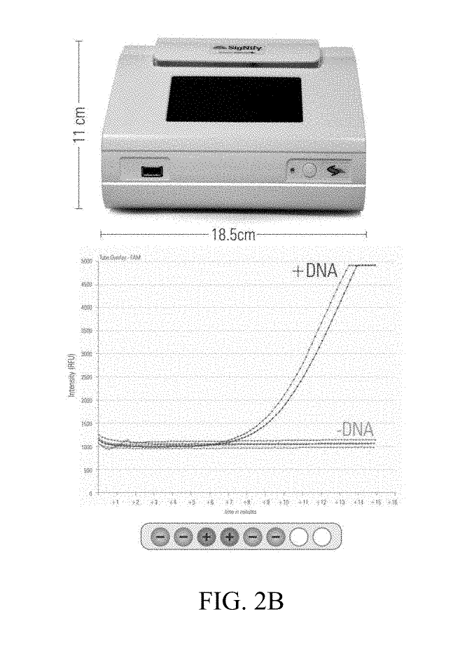

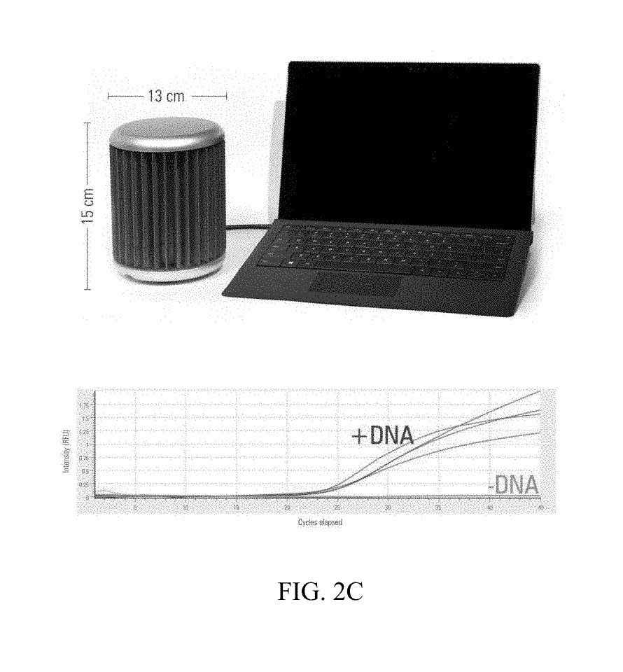

Acetaminophen was marked with a DNA mark at a local over-the-counter generics pharmaceutical manufacturer on Long Island, N.Y. in August 2014. A 5 mL DNA concentrate (DNA, water and proprietary food grade/pharmaceutical grade surfactant(s)) was then inoculated into one liter of OPACODE S-1-17823 black ink, and run through a R. W. Hartnett, Model B-15-8 printer for direct printing onto Acetaminophen capsules. Samples of non-marked Acetaminophen were purchased off the shelf from local pharmacies. As seen in the top left panel in FIG. 1, there is virtually no difference in appearance between the two capsules. None of the DNA marked capsules have been released outside of this trial.

### Sampling Methods

Capsules were swabbed by a general use Puritan 6″ Sterile Tapered Mini-tip Cotton Swab w/Wooden Handle (Puritan REF. 25-8265WC), normally used by medical professionals, engineers, and artists, wet by a solvent such as water, ethanol, isopropanol, methyl ethyl ketone with equivalent results, though the results shown in FIGS. 2A-2C utilized only ethanol. These solvents do not dissolve or damage the capsule, only the marked ink. The tips of these swab samples were then clipped for direct application into the 0.2 mL or 0.1 mL reaction tube. As seen in FIG. 1, the swab samples were clipped into 0.2 mL strip tubes.

Pre-Screening utilizing PCR and CE Methods

PCR thermocyclers utilized for the PCR-CE tests are either the Applied Biosystems 2720 Thermal Cycler (catalogue number: 4359659) or SimpliAmp™ Thermal Cycler by Thermo Fisher (catalogue number: A24811). The PCR Master Mix contains: Extract-N-Amp PCR ReadyMix™ (Sigma-Aldrich product number: E3004), Extraction Solution (Sigma-Aldrich product number: E7526), Dilution Solution (Sigma-Aldrich product number: D5688), 25 mM MgCl₂ (New England Biolabs® Inc. catalogue number: B9021S), PCR Certified Water (Teknova category number: W3330). The primers were purchased from Integrated DNA Technologies, where the forward primer was labeled with FAM or HEX. For the displayed electropherograms in FIG. 2A, HEX labeled primers were used, though to keep the figure colors consistent, the color DNA containing traces were changed to blue from green. The thermocycling parameters were 1 round at 95.0° C. for 3 minutes followed by 32 cycles of 94.0° C. denature for 20 sec, anneal at 48.0° C. for 20 sec, and elongation at 72.0° C. for 20 sec. This is followed by a final elongation step at 72.0° C. for 5 min and a 4.0° C. hold until the operator can get to the machine.

For capillary electrophoresis (CE), the Applied Biosystems Instruments 3130xL (catalog number: 3130XL) and the 3500xL (catalog number: 4440471) were both used interchangeably with equivalent results. Polymer Pop-7 (catalogue number for 3130xL: 4352759; catalogue number for 3500xL: 4393714) was used with the 36 cm array (catalogue number for 3130xL: 4352759; catalogue number for 3500xL: 4404687), using 1× Genetic Analyzer Buffer with EDTA (Gel Company number: DAB-01) and de-ionized water. The analysis solution per well includes 10 μL of HiDi (Thermo Fisher catalog number: 4311320), 0.125 μL of Liz 600 size standard (Thermo Fisher catalog number: 4408399), with 1 μL of PCR product. The instrument used for the electropherograms in FIG. 2A was the 3130xL, though the image of the CE was the 3500xL. The instrumentation and results are representatively summarized in FIG. 2A.

Real Time Quantitative PCR (qPCR) and Detection Thereof

qPCR analysis was conducted after the DNA results of negatives and positives were confirmed. The qPCR reagents, TaqMan® Fast Advanced Master Mix Catalog number: 4444963, were purchased from Thermofisher; also a GE Life Sciences, illustra PuReTaq Ready-To-Go PCR Beads Product code: 27-9559-01 was evaluated as well. The amplification procedure was performed using the reagents and protocols from the vendor. The TaqMan™ Probe and primer mix was manufactured by Thermofisher using their Custom TaqMan® Gene Expression Assay, Catalog number: 4331348.

The device utilized in this publication was the MyGo Mini, from IT-IS Life Science Ltd. IT-IS Life Science Ltd also provides a larger desktop version of the MyGo Mini called the MyGo Pro. The MyGo pro can handle 32 samples, double that of the MyGo Mini, and it has an open tangential optical path which makes it suitable for direct analysis of swabs and other solid materials, not seen in many lab bench qPCR devices.

The qPCR were performed according to specified instructions provided by the kits provided. For each sample, using the GE Pellet process, the master mix was used for re-suspension of two freeze-dried reagent pellets in frosted 0.1 mL flip cap tubes with a 50 μL of reagents total volume per tube, the composition of which are below in Supplementary Table 2.

SUPPLEMENTARY TABLE 2
The composition of the Master Mix for the GE illustra
PuReTaq Ready-To-Go PCR Beads (Panel A) and
Thermo Fisher's FastTaq Ready Mix (Panel B).

| Panel A: GE illustra | | | Panel B: Thermo Fisher's | | |
|---|---|---|---|---|---|
| PuReTaq Ready-To-Go PCR Beads | | | FastTaq Ready Mix | | |
| PCR reaction | | | PCR reaction | | |
| mix component | Per Tube | | mix component | Per Tube | |
| Custom TaqMan ® | 1.5 | μL | Custom | 6.25 | μL |
| Gene Expression Assay | | | TaqMan ® Gene | | |
| | | | Expression Assay | | |
| PCR Grade H20 | 48.5 | μL | PCR Grade H20 | 18.75 | μL |
| GE illustra PuReTaq | 2 | pellets | FastTaq | 25 | μL |
| Ready-To-Go PCR | | | | | |
| Beads | | | | | |
| Total Volume | 50 | μL | Total Volume | 50 | μL |

The sample used for analysis consists of cutting off the tip of a cotton swab after swabbing of the marked pharmaceutical. The reaction then underwent thermocycling utilizing a MyGo Mini for 40 cycles of a 2 cycle PCR at 95° C. for 10 sec and 60° C. for 30 sec, with the acquisition taking place in the 60° C. This assay takes approximately one hour for the amplification and detection process. The instrumentation and results are representatively summarized in FIG. 2C.

FIG. 2C shows the results utilizing the MyGo mini and the GE master mix reagents with ethanol swabs. Additional results not published here included: MyGo mini and the Thermo Fisher master mix reagents with ethanol swabs, MyGo Pro and the GE master mix reagents with ethanol swabs, and MyGo pro and the Thermo Fisher master mix reagents with ethanol swabs.

### Recombinase Polymerase Amplification (RPA) and Detection Thereof

RPA analysis was conducted after the DNA results of negatives and positives were confirmed. The RPA kit was purchased from TwistDx. The RPA isothermal amplification procedure was performed using the reagents and protocols from the TwistAmp exo kit. A custom TwistAmp exo assay was developed by ADNAS and then sent to TwistDx to customize a freeze dried kit as summarized in Supplementary Table 3.

SUPPLEMENTARY TABLE 3
The composition of the Master Mix for the Customized TwistAmp
exo assay

| TwistAmp exo assay | | Off the Shelf | Custom Assay |
|---|---|---|---|
| reaction mix component | | Per Tube (μL) | Per Tube (μL) |
| Custom | Standard Freeze | N/A | N/A |
| Freeze | Dried Pellet Components | | |
| Dried | 10 μM AF Primer | 4.2 | N/A |
| Pellet | 10 μM AR Primer | 4.2 | N/A |
| | 10 μM D Primer | 4.2 | N/A |
| | 10 μM Exo probe | 1.2 | N/A |
| Custom | Rehydration Buffer | 29.5 | 29.5 |
| Buffer | PCR Grade H20 | 0.7 | 14.5 |
| | 280 mM MgAc | 6 | 6 |
| | Total Volume | 50 | 50 |

This kit is then developed into a two part system: a vacuum sealed pouch with 0.2 mL tubes containing custom freeze dried pellets and a tube of custom buffer, where both parts do not require refrigeration. The swab tip is cut into the 0.2 mL reaction tube containing the custom freeze dried Pellet and then rehydrated with 50 μL of the custom buffer, as per their protocols in their standard TwistAmp exo kit. The reaction is then incubated in an Axxin T8-ISO at 38° C. for 15 min. Fluorescence measurements were taken every 26 seconds. The instrumentation and results are representatively summarized in FIG. 2B.

The Axxin device can interpret and visualize in real time the amplification process on the same device. There is software available from Axxin to program algorithms for analysis for machine interpretation and for more detailed analysis on a PC or Tablet. They have also developed a networking capability to send data from the field back to a server for data storage and analysis. This device can be either plugged into a wall socket, a car, or an external battery pack.

A similar instrument made by Axxin is the T16-ISO. The instrument is a larger unit that can be used the same way as the Axxin T8-ISO, with double the capacity. An interesting finding is that the TwistDx assay can also be utilized in the MyGo mini and the MyGo Pro, where the thermocycling temperatures have been set at 38° C. The timing of test results are similar to the Axxin T8-ISO, though the cycling parameters were set to 40 cycles. The other reason why the MyGo instruments were not focused on for these assays was that they lack the machine interpretation found on the Axxin T8-ISO and T16-ISO machines.

1. A method of authenticating a pharmaceutical product, the method comprising:
adding a detectable nucleic acid marker to a coating to form a nucleic acid-marked coating;
coating a pharmaceutical product with at least a portion of the nucleic acid-marked coating to form a tagged pharmaceutical product;
obtaining a sample from the tagged pharmaceutical product; and
detecting the presence of the detectable nucleic acid marker in the sample to authenticate the pharmaceutical product.

2. The method according to claim 1, wherein the detection is conducted with an in-field nucleic acid detection device.

3. The method according to claim 2, wherein the presence of the detectable nucleic acid marker in the sample is detected without extraction or purification of the sample.

4. The method according to claim 3, wherein detecting the presence of a nucleic acid marker in the sample is done using isothermal amplification and a sequence specific detection technique.

5. The method according to claim 3, wherein detecting the presence of the detectable nucleic acid marker in the sample is done using RPA and an intercalating dye.

6. The method according to claim 3, wherein detecting the presence of the detectable nucleic acid marker in the sample is done using PCR-based techniques selected from the group consisting of qPCR; and qPCR and an intercalating dye.

7. The method according to claim 3, wherein the in-field nucleic acid detection device is an integrated system, a microarray, or a next-generation DNA sequencer.

8. The method according to claim 3, wherein detecting the presence of the detectable nucleic acid marker in the sample is done using PCR-CE.

9. The method according to claim 3, wherein detecting the presence of the detectable nucleic acid marker in the sample is done using a sequence selective probe qPCR assay.

10. The method according to claim 1, wherein the sample is subjected to an amplification reaction to produce one or more amplification products that are characteristic of the nucleic acid marker.

11. The method according to claim 10, wherein the amplification is performed by Multiple Annealing and Loop based amplification (MALBAC), Strand Displacement amplification (SDA), Nicking Enzyme amplification reaction (NEAR), Recombinase Polymerase amplification (RPA), Helicase dependent amplification (HDA), Thermal Helicase dependent amplification (tHDA), Loop Mediated isothermal amplification (LAMP), or quantitative PCR (qPCR).

12. The method according to claim 1, wherein the detectable nucleic acid marker is a detectable DNA marker.

13. The method according to claim 12, wherein the detectable DNA marker is present in the pharmaceutical product in a concentration of less than about 1 μg/mL.

14. The method according to claim 12, wherein the detectable DNA marker is about 20 bp to about 700 bp in length and is present in the pharmaceutical product in a quantity of less than about 1×10⁻¹² g.

15. The method according to claim 1, wherein the coating is a film.

16. The method according to claim 1, wherein the coating comprises sugar, natural polymers, or synthetic polymers.

17. The method according to claim 1, wherein the coating is an enteric coating to delay release of an active pharmaceutical ingredient within the pharmaceutical product.

18. The method according to claim 1, wherein the coating comprises a colorant.

19. The method according to claim 1, wherein the pharmaceutical product is a tablet.

20. The method according to claim 1, wherein the pharmaceutical product is a capsule.
